(19) United States
(12) Patent Application Publication
(10) Pub. No.: US 2019/0059705 A1
(43) Pub. Date: Feb. 28, 2019

(54) OVERTUBE AND MEDICAL SYSTEM

(71) Applicant: Olympus Corporation, Tokyo (JP)

(72) Inventor: Takumi Isoda, Tokyo (JP)

(73) Assignee: Olympus Corporation, Tokyo (JP)

(21) Appl. No.: 16/171,494

(22) Filed: Oct. 26, 2018

Related U.S. Application Data
(63) Continuation of application No. PCT/JP2017/009719, filed on Mar. 10, 2017.

(51) Int. Cl. A61B 1/00; A61B 1/018; A61B 1/01; A61B 17/29

(52) U.S. Cl. CPC A61B 1/00135; A61B 1/018; A61B 1/00154; A61B 1/0125; A61B 1/00133; A61B 17/29; A61B 1/01

(57) ABSTRACT

The technology disclosed herein is directed to a medical system having an endoscope, a medical manipulator, an operation input device, and an overtube used to protect the endoscope during an operation. The overtube includes an elongated distal part, an elongated proximal part, and a channel. The elongated distal part is to be inserted into a body cavity of a patient. The elongated proximal part is connected to a proximal end side of the distal part. The channel is configured to extend in a longitudinal direction through the respective distal and proximal parts and to permit insertion of a medical manipulator thereinto. The proximal part contains therein at least a portion of an expandable or bendable portion configured to be expandable in the longitudinal direction and to be bendable in a direction intersecting the longitudinal direction.

### CROSS-REFERENCE TO RELATED APPLICATIONS

This application is a continuation application of PCT Application No. PCT/JP 2017/009719 filed on Mar. 10, 2017, which is hereby incorporated by reference in its entirety.

### TECHNICAL FIELD

The technology disclosed herein relates generally to an endoscopic system, and more particularly, some embodiments relate to a combination of an overtube and a medical system.

### DESCRIPTION OF THE RELATED ART

A mother-baby endoscope is known in the art. The mother-baby endoscope includes first endoscope, or a mother scope, and a second endoscope, or a baby scope inserted in a guide channel of the mother scope. As disclosed in a Japanese Patent JP 4040169 B2, the first endoscope is to be placed in a body cavity for the observation of an affected part. In this mother-baby endoscope, there is a need to allow a distal end of the baby scope to project from a distal end of the mother scope for conducting an observation through the baby scope. Stated specifically, the baby scope is attached to an inlet of the guide channel of the mother scope by a baby scope holder. The baby scope holder is configured to be expandable or contractible, so that the baby scope holder has a greater amount of expansion or contraction than an amount of projection of the baby scope from the distal end of the mother scope. As a consequence, the baby scope can be pulled into the guide channel from the distal end of the mother scope by causing an expansion of the baby scope holder. In other words, the baby scope is not restrained at a proximal end side thereof in the mother-baby endoscope of as disclosed in the Japanese Patent JP 4040169 B2, and therefore the baby scope can be accommodated in the guide channel of the mother scope by causing a retraction of the baby scope relative to the mother scope.

### BRIEF SUMMARY OF EMBODIMENTS

In one aspect of the technology disclosed herein is directed to a medical system having an endoscope, a medical manipulator, an operation input device, and an overtube used to protect the endoscope during an operation. The overtube includes an elongated distal part, an elongated proximal part, and a channel. The elongated distal part is to be inserted into a body cavity of a patient. The elongated proximal part is connected to a proximal end side of the distal part. The channel is configured to extend in a longitudinal direction through the distal part and the proximal part and to permit insertion of a medical manipulator thereinto. The proximal part contains therein at least a portion of an expandable/bendable portion configured to be expandable in the longitudinal direction and to be bendable in a direction intersecting the longitudinal direction.

Another aspect of the disclosed technology is directed to an overtube comprises an elongated distal part configured to be inserted into a body cavity of a patient. An elongated proximal part is connected to a proximal end side of the elongated distal part. A channel is configured to extend in a longitudinal direction through the elongated distal part and the elongated proximal part so as to insert a medical manipulator thereinto. The proximal part includes an expandable/bendable portion configured to be expandable in the longitudinal direction and to be bendable in a direction intersecting the longitudinal direction.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 illustrates a top view of a medical system used to operate on a body of a patient according to one embodiment of the technology disclosed herein.

FIG. 2 is a perspective view illustrating some parts of the medical system depicted in FIG. 1.

FIG. 3 is an enlarged perspective view illustrating examples of a portion of medical manipulator and endoscopes exposed at a distal end of an overtube according to the embodiment as incorporated in the medical system of FIG. 1.

FIG. 4A is a vertical cross-sectional view illustrating the overtube of FIG. 3 and the medical manipulator inserted inside the overtube.

FIG. 4B is a vertical cross-sectional view illustrating a state that a distal end of the medical manipulator is accommodated in a channel of the overtube of FIG. 4A with the overtube being expanded and bent.

FIG. 5A is a vertical cross-sectional view illustrating a modification of FIG. 4A.

FIG. 5B is a vertical cross-sectional view illustrating a state that the distal end of the medical manipulator is accommodated in a channel of the overtube of FIG. 5A with the overtube being expanded and bent.

FIG. 6A is a vertical cross-sectional view illustrating another modification of FIG. 4A.

FIG. 6B is a vertical cross-sectional view illustrating a state that the distal end of the medical manipulator is accommodated in a channel of the overtube of FIG. 6A with the overtube being expanded and bent.

FIG. 7 is a perspective view illustrating a modification of the medical system depicted in FIG. 2.

### DETAILED DESCRIPTION OF THE EMBODIMENTS

In the following description, various embodiments of the technology will be described. For purposes of explanation, specific configurations and details are set forth in order to provide a thorough understanding of the embodiments. However, it will also be apparent to one skilled in the art that the technology disclosed herein may be practiced without the specific details. Furthermore, well-known features may be omitted or simplified in order not to obscure the embodiment being described.

In the prior art, if the baby scope is restrained at the proximal end side thereof or if no space is available at the proximal end side of the baby scope to permit its retraction, then, the baby scope is not permitted to retract relative to the mother scope. To accommodate the baby scope in the guide channel, it is therefore necessary to allow the mother scope to advance relative to the attached baby scope, whereby an inconvenience arises in that the visual field of the mother scope varies.

Accordingly, there is a need for an overtube and a medical system that, even if a medical manipulator movably inserted in a channel is restrained at a proximal end side thereof or even if no space is available at the proximal end side of the medical manipulator to permit its retraction, a distal end of the medical manipulator, the distal end projecting from a distal end of the overtube, can be retracted and pulled into the channel.

In one aspect of the technology disclosed herein is directed to a medical system having an endoscope, a medical manipulator, an operation input device, and an overtube used to protect the endoscope during an operation. The overtube includes an elongated distal part, an elongated proximal part, and a channel. The elongated distal part is to be inserted into a body cavity of a patient. The elongated proximal part is connected to a proximal end side of the distal part. The channel is configured to extend in a longitudinal direction through the distal part and the proximal part and to permit insertion of a medical manipulator thereinto. The proximal part contains therein at least a portion of an expandable/bendable portion configured to be expandable in the longitudinal direction and to be bendable in a direction intersecting the longitudinal direction. According to this aspect, the elongated distal part of the overtube is inserted into the body cavity of the patient. The elongated proximal part, which is connected to the proximal end side of the distal part, is disposed outside the body of the patient. The medical manipulator is inserted and driven in the channel included through the distal part and proximal part. Treatment is conducted on a tissue in the body cavity by the medical manipulator projecting forward of the distal part of the overtube. The expandable/bendable portion is located in the proximal part of the overtube. At this position, the expandable/bendable portion is expanded in the longitudinal direction while allowing the expandable/bendable portion to bend in the direction intersecting the longitudinal direction. Then, a distal end of the medical manipulator is moved toward the proximal end thereof relative to the overtube, and pulled into the channel without movement of a proximal end of the medical manipulator.

In the herein-described aspect, the expandable or bendable portion may include an expandable portion and a bendable portion. The expandable portion is expandable in the longitudinal direction. The bendable portion is disposed in series in the longitudinal direction with the expandable portion and the bendable portion is bendable in the direction intersecting the longitudinal direction. When configured as noted herein, by causing a bending of the bendable portion while allowing the expandable portion to expand, the distal end of the medical manipulator is moved toward the proximal end thereof relative to the overtube, and pulled into the channel without movement of the proximal end of the medical manipulator. The overtube may further include attaching part for permitting switching between two states. One state is that the expandable portion is attached to avoid an expansion or contraction. The other state is that the expandable portion is allowed to expand. When configured as described herein, by actuating the attaching part to keep the expandable portion attached without an expansion, it is possible to obviate the occurrence of inconvenience that the expandable portion undergoes an unintentional expansion upon insertion or the like of the medical manipulator into the channel. By switching the attached part to make the expandable portion expandable when needed, the distal end of the medical manipulator is moved toward the proximal end side thereof relative to the overtube, and pulled into the channel without movement of the proximal end of the medical manipulator. It should be noted that the expandable portion may be a bellows. The proximal part of the overtube is allowed to readily expand and contract by expansion and contraction of the bellows. The overtube may further include, inside the expandable portion, an inner tube having a length to cover an inner wall of the expandable portion in a contracted state thereof and attached at an end thereof on one of opposite ends of the expandable portion. When configured as described, in a state that the bellows is contracted, the inner tube covers the inner wall of the bellows, so that during insertion of the medical manipulator into the channel, the medical manipulator can be prevented from being caught on the inner wall of the bellows and can be smoothly inserted.

In another aspect of the technology disclosed herein, a medical system includes any one of the hereinbefore-described overtubes, a medical manipulator, and a drive unit. The medical manipulator is to be inserted into the channel of the overtube. The medical manipulator includes a proximal part that extends from a proximal end side of a proximal part of the overtube. The drive unit is configured to permit connection of the proximal part of the medical manipulator to the drive unit, and to drive the medical manipulator. The elongated distal part of the overtube is inserted into the body cavity of the patient. The elongated proximal part is remained outside the body of the patient. The elongated proximal part is connected to the proximal end side of the distal part. The proximal part of the medical manipulator is connected to the drive unit. The medical manipulator is inserted in the channel formed through the distal part and proximal part. The drive unit is actuated. Then treatment can be conducted on a tissue in the body cavity of the patient by the medical manipulator projecting forward of the distal part of the overtube. Here, the expandable/bendable portion is included in the proximal part of the overtube. The proximal part of the medical manipulator is connected to the drive unit. The expandable/bendable portion is expanded in the longitudinal direction while bending the expandable/bendable portion in the direction intersecting the longitudinal direction. The distal end of the medical manipulator is moved toward the proximal end side thereof relative to the overtube, and pulled into the channel without movement of the drive unit in the longitudinal direction. The medical system may further include a locking portion configured to lock the expandable/bendable portion in an expanded state. When configured as described herein, by locking the expandable/bendable portion in the expanded state with the lock portion, the distal end of the medical manipulator is maintained in a state that the distal end of the medical manipulator is accommodated in the channel of the overtube, whereby the medical manipulator is prevented from acting as an obstruction upon moving the overtube. The technology disclosed herein brings about at least some advantageous effects that, (i) even if the medical manipulator movably inserted in the channel is restrained at the proximal end side thereof or (ii) even if no space is available at the proximal end side of the medical manipulator to permit its retraction, the distal end of the medical manipulator, the distal end projecting from the distal end of the overtube, is retracted and pulled into the channel.

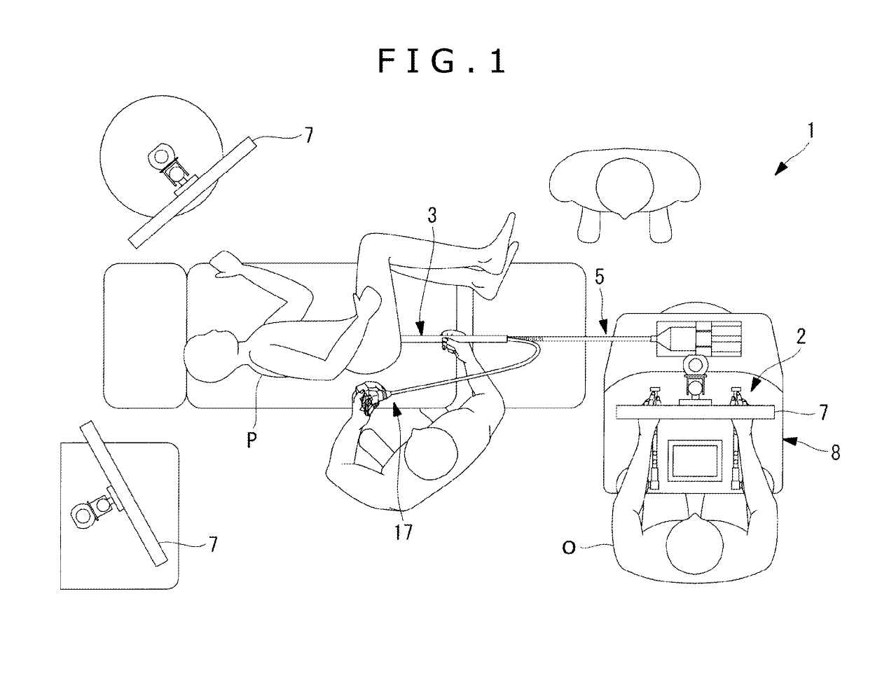

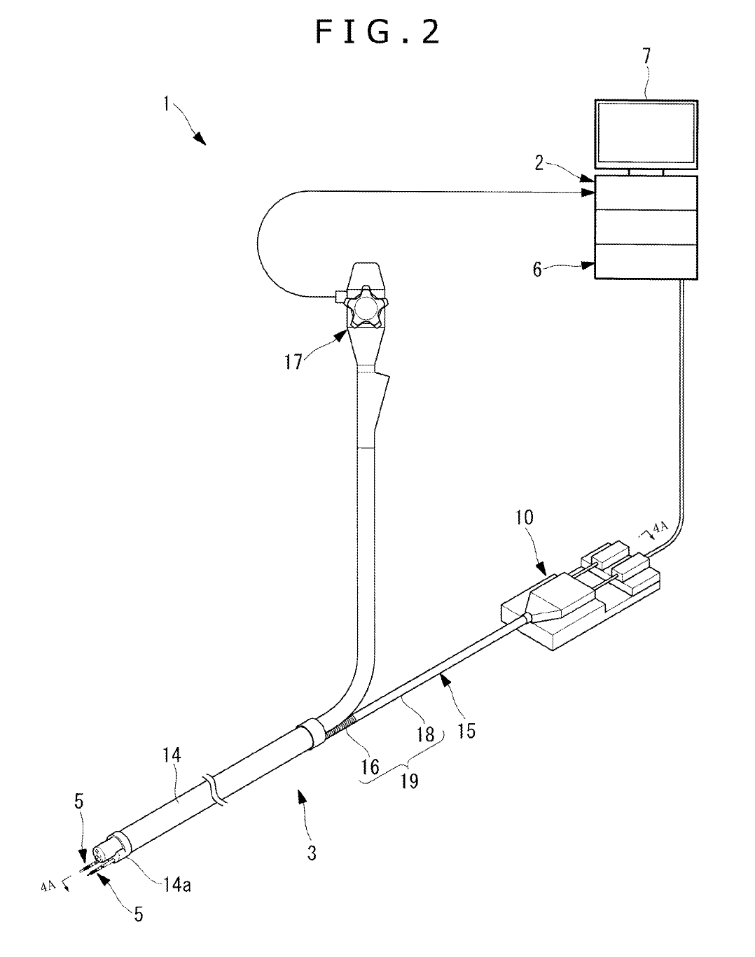

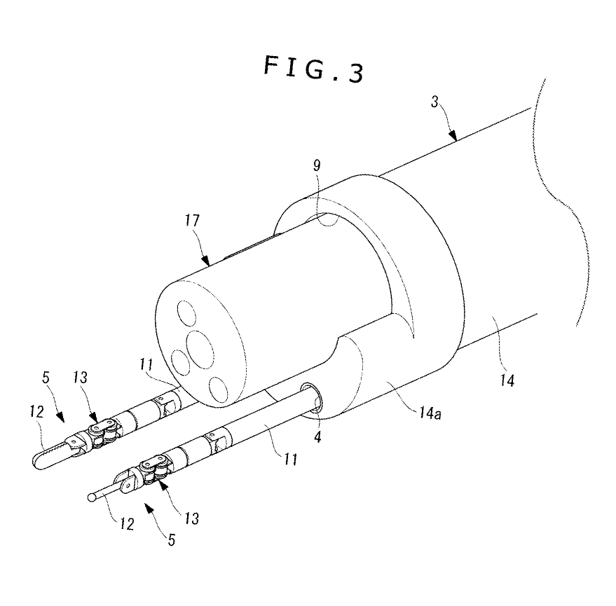

As illustrated in FIGS. 1 to 3, the medical system 1 includes as an operation input device 2, an overtube 3, two medical manipulators 5, an endoscope 17, a drive unit 10, a controller 6, a monitor 7, and a console 8. The operation input device 2 is to be operated by an operator (0). The overtube 3 is to be inserted into a body cavity of a patient (P). The two medical manipulators 5 are to be inserted into two respective channels 4 of the overtube 3. It should be noted that in FIG. 3 one of two channels 4 is viewable in which the other channel 4 is blocked from viewing by the endoscope 17. The endoscope 17 is to be inserted into a channel 9 of the overtube 3. The drive unit 10 drives the medical manipulators 5 based on operations of the operation input device 2. The controller 6 controls the drive unit 10. The operation input device 2, drive unit 10, controller 6 and monitor 7 are supported by the console. As illustrated in FIG. 3, the medical manipulators 5 each includes a slender soft part 11, a surgical device 12, and a joint 13. The surgical device 12, for example, but not limiting, is grasping forceps arranged on a distal end of the soft part 11. The joint 13 changes the orientation of the surgical device 12.

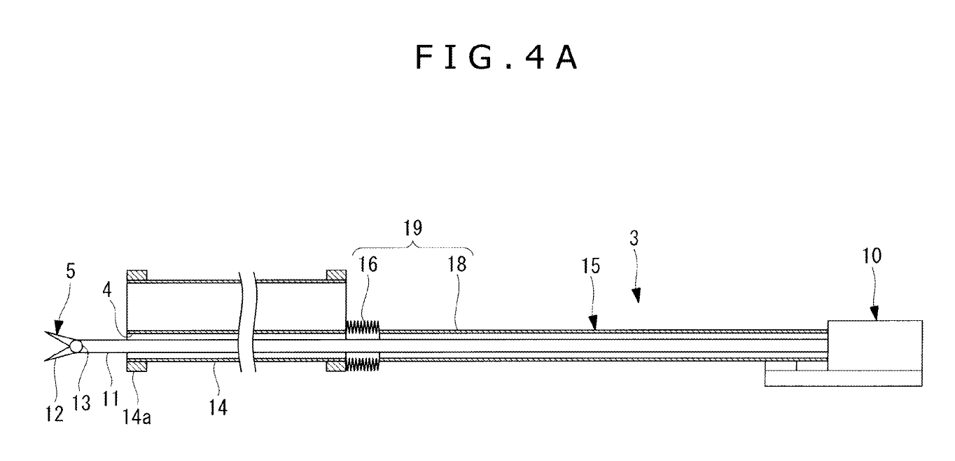

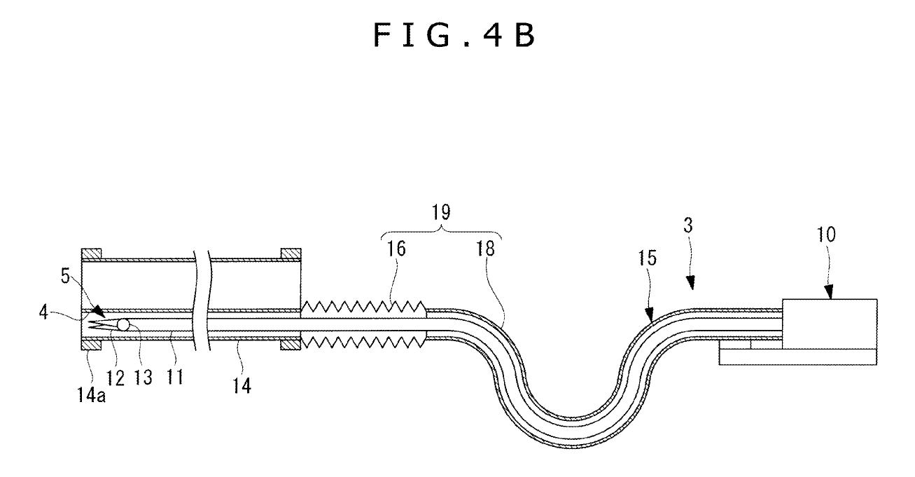

As illustrated in FIG. 4A, the drive unit 10 is connected to proximal ends of the soft parts 11, and is configured to generate power, which drives the medical manipulators 5, responsive to instructions from the controller 6 based on operations through the operation input device 2. The drive unit 10 includes an unillustrated electric motor that generates power, and is configured to actuate each surgical device 12 and its associated joint 13 via a corresponding power transmission member such as a wire by rotation of the electric motor. As illustrated in FIGS. 1 and 4A, the overtube 3 includes an elongated distal part 14 and an elongated proximal part 15. The distal part 14 is arranged on a distal end side and is to be inserted into the body of the patient (P). The proximal part 15 is connected to a proximal end side of the distal part 14 and is to be remained outside the body of the patient. The respective channels 4 are arranged extending in a longitudinal direction through the distal part 14 and proximal part 15. The proximal part 15 to be remained outside the body of the patient includes, in a portion thereof, an expandable/contractible/bendable portion 19, or expandable/bendable portion. The expandable/contractible/bendable portion 19 is formed of an expandable/contractible portion 16, or expandable portion, and a bendable portion 18 arranged in series. The expandable/contractible portion 16 expands/contracts, expands in the longitudinal direction. Numeral reference 14a designates a cap arranged on a distal end of the distal part 14. The distal part 14 includes the two channels 4 and the single channel 9. The respective medical manipulators 5 are to be inserted into the two channels 4. The endoscope is to be inserted into the single channel 9. The proximal part 15, on the other hand, includes the two channels 4 into which the respective medical manipulators 5 are to be inserted. As illustrated in FIGS. 4A and 4B, the expandable/contractible portion 16 is formed in shape of a bellows. The bendable portion 18 is formed in shape of a rubber tube, for example. Accordingly, when the operator (O) applies external force to the bendable portion 18 in the direction intersecting the longitudinal direction, the bendable portion 18 is bent, and at the same time, the expandable/contractible portion 16 expands, whereby the overall length of the overtube 3 can be increased. Here, the proximal ends of the respective medical manipulators 5 are attached to the drive unit 10 and therefore are immovable, and further the overall lengths of the medical manipulators 5 remain unchanged. It is, therefore, the distal ends of the medical manipulators 5, the distal ends projecting forward of a distal end of the overtube 3, are pulled into the corresponding channels 4 of the overtube 3.

A description will hereinafter be made about operations of the overtube 3 and medical system 1 according to the embodiment configured as described hereinbefore. To treat an affected part in the body cavity of the patient (P) by using the medical system 1, the distal part 14 of the overtube 3 is inserted into the body cavity of the patient (P). The medical manipulators 5 are inserted into the respective channels 4 from the proximal end of the proximal part 15, the proximal end is remained outside of the body of the patient (P). The surgical devices 12 on the distal ends of the respective medical manipulators 5 are allowed to project forward from the distal end of the overtube 3. The proximal ends of the respective medical manipulators 5 extend rearward from the proximal end of the proximal part 15 of the overtube 3. Then the proximal ends of the respective medical manipulators 5 are connected to the drive unit 10. By actuating the drive unit 10 based on signals from the controller 6 in this state, the joints 13 and surgical device 12 on the distal ends of the respective medical manipulators 5 are actuated to enable the treatment of the affected part of the patient.

If desired to change the position of the distal end of the overtube 3 in the course of the treatment, the distal ends of the medical manipulators 5 is pulled into the corresponding channels 4 in some instances. Here, it is to be noted that the distal end of the overtube 3 is moved in two ways, one being a movement by advancement or retraction, and the other a movement through the bending action. The movement through bending action is performed by an unillustrated wire, or is performed using a bent part of the endoscope 17 inserted in the other channel 9. In this embodiment, the operator (O) can apply external force in the direction intersecting the longitudinal direction to the bendable portion 18 in the expandable/contractible/bendable portion 19. The expandable/contractible/bendable portion 19 is included in the proximal part 15 disposed outside the body of the patient (P). The proximal part 15 is partially bent at a portion thereof. As a consequence, the bendable portion 18 which has been straight before is now bent, so that the distance between opposite ends of the bendable portion 18 decreases and the expandable/contractible portion 16 is hence allowed to expand to compensate for the decrease in distance. In other words, according to the overtube 3 and medical system 1 of this embodiment, by (i) bending the bendable portion 18 of the proximal part 15 and (ii) allowing the expandable/contractible portion 16 of the proximal part 15 to expand, the overall length of the overtube 3 can be increased without a movement of both ends of the proximal part 15. In this case, the overall lengths of the respective medical manipulators 5 are remained unchanged. Therefore, the surgical devices 12 on the distal ends of the respective medical manipulators 5 can be pulled into the corresponding channels 4 of the overtube 3. As a result, the distal end of the overtube 3 can be promptly moved by pulling the distal ends of the medical manipulators 5 into the corresponding channels 4 with the proximal sides of the medical manipulators 5 being kept connected with the drive unit 10.

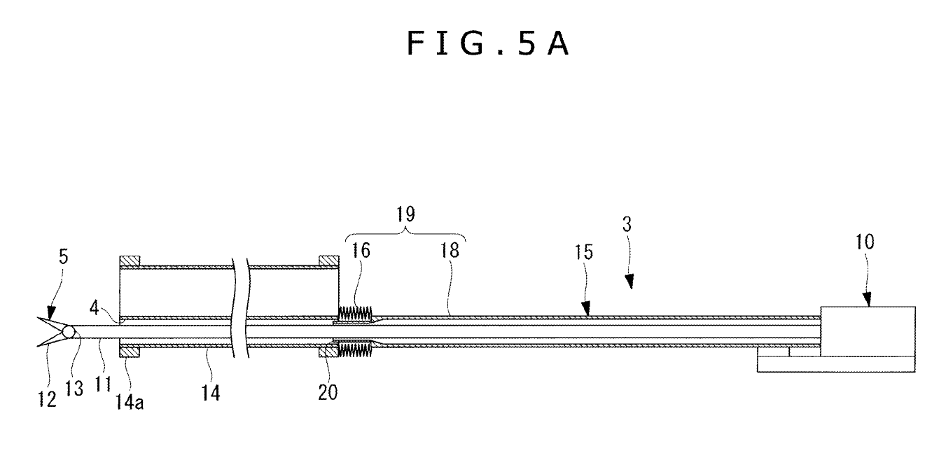

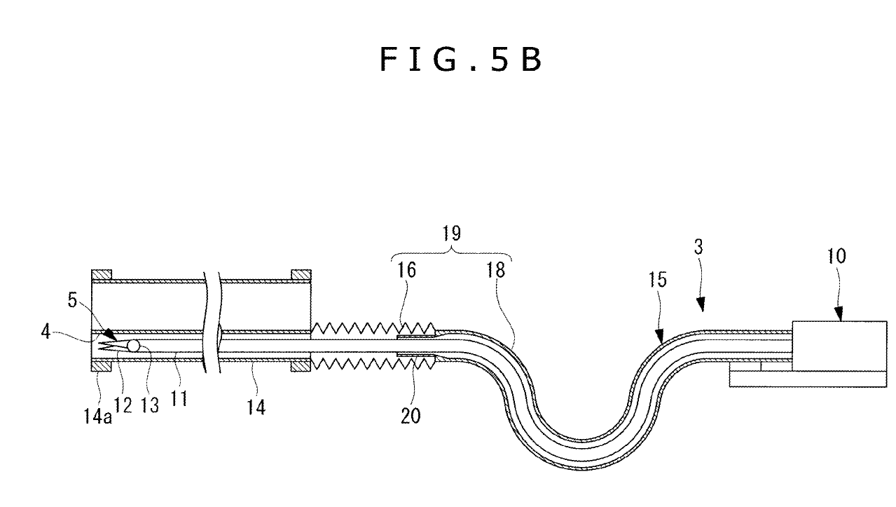

As has been described hereinbefore, the overtube 3 and medical system 1 have a merit in that the surgical devices 12 on the distal ends of the respective medical manipulators 5 can be pulled into the corresponding channels 4 of the overtube 3 (i) even if the proximal ends of the respective medical manipulators 5 cannot retract toward the sides of the proximal ends attached to the drive unit 10 or (ii) even if no spaces are available at the sides of the proximal ends to permit pulling of the distal ends into the corresponding channels 4. In this embodiment, as for each expandable/contractible portion 16, the bellows is adopted. As an alternative to the bellows, a rubber tube expandable/contractible in the longitudinal direction or a telescopically expandable/contractible structure may be adopted. Further, as for each expandable/contractible/bendable portion 19, the expandable/contractible portion 16 and the bendable portion 18 are arranged in series. As an alternative to this configuration, a rubber tube may be adopted if the rubber tube is expandable/contractible in the longitudinal direction and is bendable. As another alternative, a member may be adopted if the member can realize expansion/contraction and bending at the same time like bellows. Furthermore, in this embodiment, the expandable/contractible portion 16 and the bendable portion 18 are arranged in series in this order from the proximal end of the distal part 14 toward the side of the proximal end of the proximal part 15 of the overtube 3. However, the order of their arrangement may be reversed. If the bellows 16 is adopted as the expandable/contractible portion, an inner tube 20 may be disposed covering the inner wall of the bellows 16 that is in a contracted state. The inner tube 20 may be attached at an end thereof on one end of the bellows 16, as illustrated in FIGS. 5A and 5B. In the example illustrated in FIGS. 5A and 5B, the inner tube 20 is attached on a proximal end side of the bellows 16, but the inner tube 20 may be attached on a distal end side of the bellows 16.

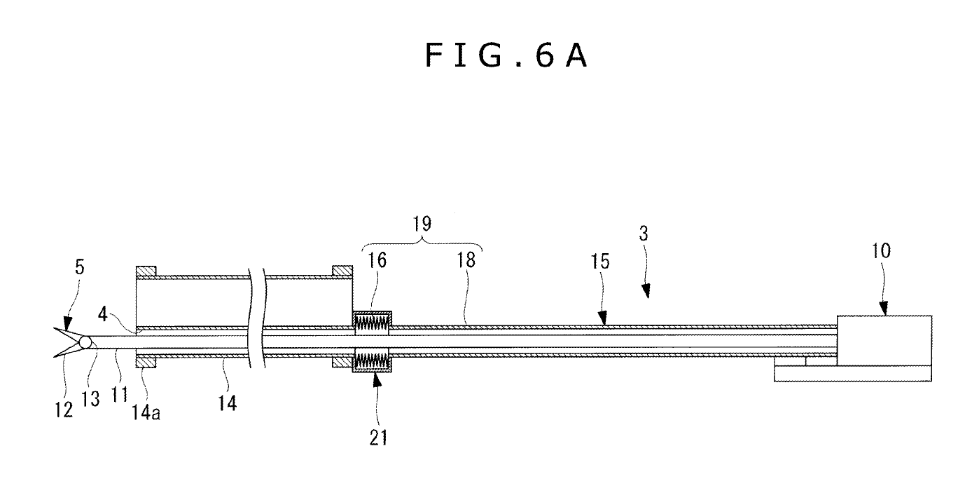

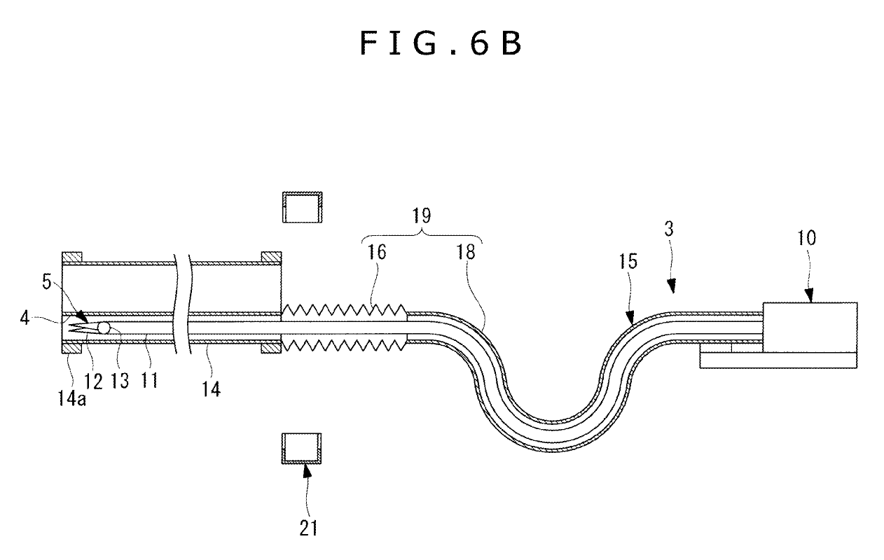

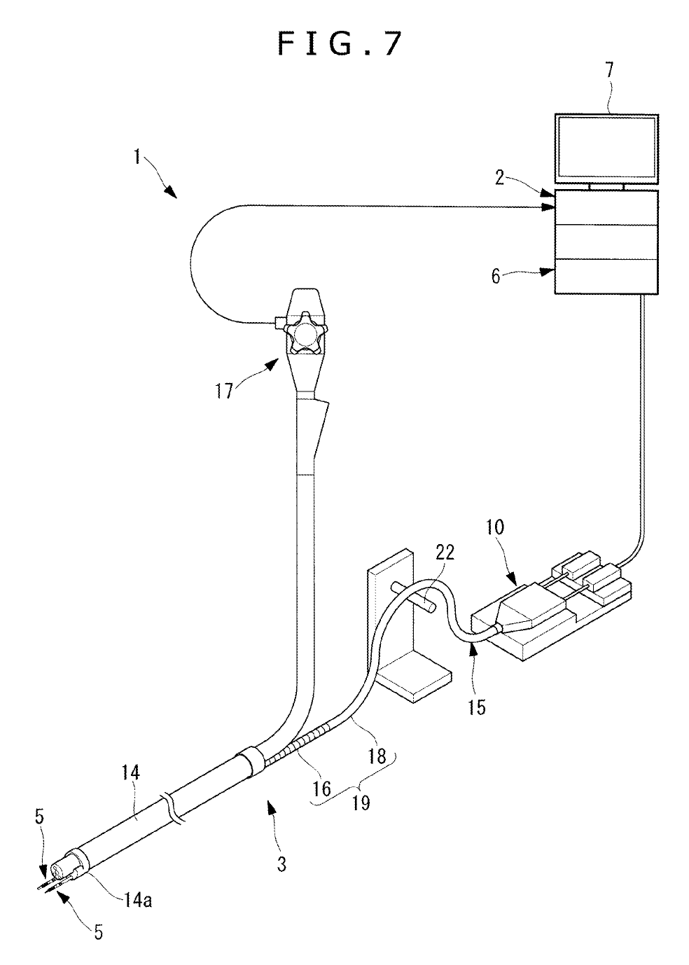

When configured as described hereinbefore, the inner wall of the bellows 16 is covered by the inner tube 20 in the state that the bellows 16 is contracted. Upon insertion of each medical manipulator 5 into the corresponding channel 4, the medical manipulator 5 can, therefore, be prevented from being caught on ridges and/or in grooves on the inner wall of the bellows 16. Upon allowing the bellows 16 is to be expanded, the insertion of the medical manipulators 5 into the corresponding channels 4 have already been completed, so that no inconvenience arises even if the inner wall of the bellows 16 becomes out of registration with the inner tube 20 and is exposed. This embodiment may further include attaching that permits switching between two states. One state is that the bellows 16 is attached in a contracted state. The other state is that bellows 16 is allowed to expand when is needed. As the attaching part, a locking mechanism 21 is adopted as illustrated in FIGS. 6A and 6B. The locking mechanism 21 is disposed detachably in a radial direction, restrains the bellows 16 from expansion in an attached state, but releases the restraint of the bellows 16 in a state that it has been detached outward in the radial direction. As an alternative to the detachable locking mechanism 21, another locking mechanism may be adopted. This locking mechanism is disposed slidably, restrains the bellows 16 from expansion in a state that the locking mechanism has moved inwards in the radial direction, but releases the restraint of the bellows 16 in a state that the locking mechanism has moved outward in the radial direction. As an alternative to the slide mechanism, a pivotal mechanism may be adopted. Further, any desired locking mechanism may be adopted. In addition, this embodiment may also include locking part 22, or a locking portion, which locks the bellows 16 in an expanded state. As the locking part 22, it is possible, as illustrated in FIG. 7, to adopt a hook or the like which is disposed in a vicinity of the overtube 3. The bendable portion 18 can be hooked on the locking part 22 in a bent state. In view of the inclusion of the locking part 22, the distal ends of the respective medical manipulators 5 can be maintained in a state that they have been pulled in the corresponding channels 4 of the overtube 3, so that the position of the distal end of the overtube 3 can be readily changed.

In sum, the disclosed technology is directed to an overtube comprises an elongated distal part configured to be inserted into a body cavity of a patient. An elongated proximal part is connected to a proximal end side of the elongated distal part. A channel is configured to extend in a longitudinal direction through the elongated distal part and the elongated proximal part so as to insert a medical manipulator thereinto. The proximal part includes an expandable/bendable portion configured to be expandable in the longitudinal direction and to be bendable in a direction intersecting the longitudinal direction. The expandable/bendable portion includes an expandable portion expandable in the longitudinal direction. A bendable portion is disposed in series in the longitudinal direction with the expandable portion and bendable in the direction intersecting the longitudinal direction.

The overtube further comprises an attaching part configured to switch between a contracted state and an expanded state, wherein the expandable portion is fixed to avoid an expansion or contraction in the contracted state, and the expandable portion is allowed to expand in the expanded state. The expandable portion is a bellows. The overtube further comprises an inner tube inside the expandable portion. The inner tube having a length to cover an inner wall of the expandable portion in the contracted state thereof. The inner tube is attached at one end thereof on one of opposite ends of the expandable portion.

The disclosed technology is further directed to a medical system comprises a medical manipulator configured to perform into a body. A drive unit configured to drive the medical manipulator. A controller is configured to control the drive unit. An overtube is configured to guide the medical manipulator into the body. The overtube comprises an elongated distal part configured to be inserted into the body. An elongated proximal part is connected to a proximal end side of the elongated distal part. The elongated proximal part is configured to be expandable and bendable. A locking portion is configured to lock the expandable/bendable portion in an expanded state. A channel is configured to extend in a longitudinal direction through the elongated distal part and the elongated proximal part so as to insert the medical manipulator thereinto. The elongated proximal part includes an expandable/bendable portion configured to be expandable in the longitudinal direction and to be bendable in a direction intersecting the longitudinal direction.

The disclosed technology is further directed to a medical system comprises an endoscope, at least one medical manipulator, a drive unit, a controller, and an elongated overtube all of which being configured to be attached to one another to perform an operation on a body of a patient. The elongated overtube comprises an elongated distal part having a proximal end side. The elongated distal part is configured to be inserted into the body of the patient. An elongated proximal part is connected to the proximal end side of the elongated distal part. A channel is configured to extend in a longitudinal direction through the elongated distal part and the proximal part so as to permit insertion of the at least medical manipulator thereinto. The proximal part includes in at least a portion thereof an expandable or bendable portion configured to be expandable in a longitudinal direction and to be bendable in a direction intersecting the longitudinal direction. The expandable or bendable portion includes an expandable portion configured to be expandable in the longitudinal direction. A bendable portion is disposed in series in the longitudinal direction with the expandable portion and bendable in the direction intersecting the longitudinal direction.

While various embodiments of the disclosed technology have been described above, it should be understood that they have been presented by way of example only, and not of limitation. Likewise, the various diagrams may depict an example schematic or other configuration for the disclosed technology, which is done to aid in understanding the features and functionality that can be included in the disclosed technology. The disclosed technology is not restricted to the illustrated example schematic or configurations, but the desired features can be implemented using a variety of alternative illustrations and configurations. Indeed, it will be apparent to one of skill in the art how alternative functional, logical or physical locations and configurations can be implemented to implement the desired features of the technology disclosed herein.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. Additionally, the various embodiments set forth herein are described in terms of exemplary schematics, block diagrams, and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular configuration.

What is claimed is:

1. An overtube comprising:
an elongated distal part configured to be inserted into a body cavity of a patient;
an elongated proximal part connected to a proximal end side of the elongated distal part; and
a channel configured to extend in a longitudinal direction through the elongated distal part and the elongated proximal part so as to insert a medical manipulator thereinto,
wherein the proximal part includes an expandable/bendable portion configured to be expandable in the longitudinal direction and to be bendable in a direction intersecting the longitudinal direction.

2. The overtube of claim 1, wherein the expandable/bendable portion includes
an expandable portion expandable in the longitudinal direction; and
a bendable portion disposed in series in the longitudinal direction with the expandable portion and bendable in the direction intersecting the longitudinal direction.

3. The overtube of claim 2, further comprising:
an attaching part configured to switch between a contracted state and an expanded state, wherein the expandable portion is fixed to avoid an expansion or contraction in the contracted state, and the expandable portion is allowed to expand in the expanded state.

4. The overtube of claim 2, wherein the expandable portion is a bellows.

5. The overtube of claim 4, further comprising:
an inner tube inside the expandable portion, the inner tube having a length to cover an inner wall of the expandable portion in the contracted state thereof, and the inner tube is attached at one end thereof on one of opposite ends of the expandable portion.

6. A medical system comprising:
a medical manipulator configured to perform into a body;
an overtube configured to guide the medical manipulator into the body;
a drive unit configured to drive the medical manipulator;
a controller configured to control the drive unit,
wherein the overtube comprising:
an elongated distal part configured to be inserted into the body;
an elongated proximal part connected to a proximal end side of the elongated distal part, the elongated proximal part is configured to be expandable and bendable; and
a channel configured to extend in a longitudinal direction through the elongated distal part and the elongated proximal part so as to insert the medical manipulator thereinto.

7. The medical system of claim 6, wherein the elongated proximal part includes an expandable/bendable portion configured to be expandable in the longitudinal direction and to be bendable in a direction intersecting the longitudinal direction.

8. The medical system of claim 7, further comprising:
a locking portion configured to lock the expandable/bendable portion in an expanded state.

9. A medical system comprising:
an endoscope, at least one medical manipulator, a drive unit, a controller, and an elongated overtube all of which being configured to be attached to one another to perform an operation on a body of a patient; and wherein
the elongated overtube comprises:
an elongated distal part having a proximal end side, the elongated distal part configured to be inserted into the body of the patient,
an elongated proximal part being connected to the proximal end side of the elongated distal part,
a channel configured to extend in a longitudinal direction through the elongated distal part and the proximal part so as to permit insertion of the at least medical manipulator thereinto, and wherein
the proximal part includes in at least a portion thereof an expandable or bendable portion configured to be expandable in a longitudinal direction and to be bendable in a direction intersecting the longitudinal direction.

10. The medical system of claim 9, wherein the expandable or bendable portion includes
an expandable portion configured to be expandable in the longitudinal direction; and
a bendable portion disposed in series in the longitudinal direction with the expandable portion and bendable in the direction intersecting the longitudinal direction.
